SEQUENCE LISTING (12 sequences)

SEQ ID NO: 1 - DNA, 43 bp, Canis lupus
atgaaggttctgtgggctgcgctggtggtcacgctcctggcag

SEQ ID NO: 2 - DNA, 211 bp, Canis lupus
gatgctgggccgatgtgcagccggagccggagctggagcgcgagctggagccgaaggtccagcaggagctggagccagaggccgggtggcagactggccagccctgggaggcggcgctggcccgcttctgggattacctgcgctgggtgcagacgctgtctgaccaggtgcaagagggcgtgctcaacacccaggtcacccaggaactgac

SEQ ID NO: 3 - DNA, 718 bp, Canis lupus
ggcgctgatggatgagaccatgaaggaggtgaaggcctacaaggcggagctggacgagcagctgggccccatgacctcggagacgcaggcccgcgtggccaaggagctgcaggcggcgcaggcccggctgcgctcggacatggaggacgtgcgcaaccgcctgacgcagtaccgcggcgagctgcaggccatgctgggccagagcagcgaggagctgcgggcgcgcttcgcctcccacatgcgcaagctgcgcaagcgggtgctgcgggacgccgaggacctgcagaggcgcctggccgtctacaaggccggcgtgcgcgagggtgccgagcgcagcgtgagcagcatccgcgagcgcctctggccgctgctggagcaggcccgcgagcgcaacgccaaggtgggcgccctggccacgcagccgctgctcgagcgggccgacgccctgggccagcagctgcgcgggcagctggaggagatgagcagccgggcccgcggccacctggaggagatgcgcgagcagatacaggaggtgcgggtgaagatggaggagcaggccgaccagatacgccaaaaggccgaggccttccaggcgcgcctcaagagctggttcgagcccctgctggaagacatgcagcgccagtgggacgggctggtggagaaggtgcaggcggccgtggccaccatccccacctctaagcctgtggaggaaccatga

SEQ ID NO: 4 - DNA, 23 bp, Canis lupus
ccgggtggcagactggccagccc

SEQ ID NO: 5 - DNA, 25 bp, Artificial Sequence
  sgRNA
atagggctggccagtctgccaccgt

SEQ ID NO: 6 - DNA, 25 bp, Artificial Sequence
  sequence complementary to sgRNA
taaaacggtggcagactggccagcc

SEQ ID NO: 7 - DNA, 17 bp, Canis lupus
  mutation (1)..(17)
cctggaccagggaggct

SEQ ID NO: 8 - DNA, 21 bp, Artificial Sequence
  forward primer
cattgttgtcaggcaggtagc

SEQ ID NO: 9 - DNA, 19 bp, Artificial Sequence
  reverse primer
gaagggtgcgagggattga

SEQ ID NO: 10 - DNA, 20 bp, Artificial Sequence
  sgRNA
gggctggccagtctgccacc

SEQ ID NO: 11 - DNA, 194 bp, Unknown
  mutation
gatgctgggccgatgtgcagccggagccggagctggagcgcgagctggagccgaaggtccagcaggagccctggaccagggaggctctgggaggcggcgctggcccgcttctgggattacctgcgctgggtgcagacgctgtctgaccaggtgcaagagggcgtgctcaacacccaggtcacccaggaactgac

SEQ ID NO: 12 - DNA, 17 bp, Canis lupus
cctggaccagggaggct

(19) United States
(12) Patent Application Publication
(10) Pub. No.: US 2018/0279591 A1
(43) Pub. Date: Oct. 4, 2018

(54) METHOD FOR PREPARING A CANINE MODEL OF ATHEROSCLEROSIS

(71) Applicant: Beijing Sinogene Biotechnology Co. Ltd., Beijing (CN)

(72) Inventors: Min Zheng, Beijing (CN); Jianping Zhao, Beijing (CN); Jidong Mi, Beijing (CN)

(21) Appl. No.: 15/727,562

(22) Filed: Oct. 6, 2017

(30) Foreign Application Priority Data
Mar. 29, 2017 (CN) 201710197156.7

(51) Int. Cl. A01K 67/027; C12N 15/85

(52) U.S. Cl. CPC A01K 67/0276; C12N 15/8509; C12N 2015/8527; A01K 2267/0375; A01K 2227/10; A01K 2217/075

(57) ABSTRACT

The present invention relates to a method for preparing a canine model of atherosclerosis, in particular, relates to a method for preparing an apolipoprotein E (APOE) gene knock-out disease canine model with the use of gene knock-out technology.

### CROSS-REFERENCE TO RELATED APPLICATION

This non-provisional application claims priority under 35 U.S.C. § 119(a) on Patent Application No. 201710197156.7, filed Mar. 29, 2017 in the State Intellectual Property Office of P.R. China, the entire content of which is hereby incorporated by reference.

### FIELD OF THE INVENTION

The present invention relates to a method for preparing a canine model of atherosclerosis, more particularly to a method for preparing an apolipoprotein E (APOE) gene knock-out disease canine model with the use of gene knock-out technology.

### BACKGROUND OF THE INVENTION

Atherosclerosis (AS) is an elderly frequently occurring disease caused by multiple factors such as inheritance and environment. It is a main cause of cardiovascular diseases such as coronary heart disease, cerebral infarction, peripheral vascular disease, etc. If patients of coronary atherosclerosis are in pipe diameter stenosis of above 75%, angina pectoris, myocardial infarction, arrhythmia, and even sudden death may occur. Cerebral atherosclerosis may cause cerebral ischemia and encephalatrophy, or lead to hemorrhage due to cerebral vascular rupture.

The etiology of AS is complicated. The incidence of AS is associated with a variety of pathogenic factors, and often occurs in large and medium-sized elastic vessels, and endometrium and underneath the endometrium of muscular arterial wall. AS is characterized by lipid deposition and endometrial thickening, and forms atherosclerosis lesions or fibrous lipid plaques. Among the multiple pathogenic factors, lipid metabolism disorder, especially hypercholesterolemia is most closely related. Apolipoprotein (Apo) E is an important component of plasma lipoproteins. It serves an important role in adjusting plasma cholesterol level and transportation and metabolism of lipid, and is an important molecular target in occurrence and development of hyperlipemia and AS, etc. Therefore, ApoE is the key in the occurrence and development of AS.

In recent years, the incidence of AS shows younger and rising tendency. It is therefore necessary to establish atherosclerosis animal models to investigate thoroughly the etiology and the medicines. Rats and mice are the most popularly used animal models, while rats are AS resistant. Further, although the formed pathologic change thereof is similar to human's early lesion, it is difficult to form later lesion similar to that of the human body, and it is not convenient to take blood from mice.

Canine is one of the commonly used laboratory animals in fundamental medical research and teaching, and especially, plays an important role in experimental researches such as physiology, pharmacology and pathophysiology. Canine also has hereditary diseases comparatively similar to that of human. Canine has less hereditary diseases, good experimental repeatability, well-developed blood circulation and nervous system. Canine is similar in digestive system and internal organs, and is closer in toxicological reactions to that of human. Therefore, canine is especially suitable for investigations in pharmacology, circulatory physiology, ophthalmology, toxicology, and surgery, etc. Further, canines are gentle and are easy to tune, and can cooperate well in experimental research through short-term training. Therefore, canines are regarded to be comparatively ideal for experimental research in international medical and biological fields.

Currently used methods for establishing a canine disease model mainly include the methods, such as feeding method, mechanical damage method, immunization method, etc. Feeding method, mechanical damage method and immunization method are to use special ways to induce healthy animals to have disease phenotypes. These methods have problems that the inductive canine animal models do not show disease phenotype, the duration of phenotype thereof is short, and the inductive canine animal models cannot simulate human disease symptoms.

The shortcomings of the above inductive canine animal models can be overcome by using gene engineering for gene knock-out or transgenic modification in non-human animals to establish disease animal models. However, the most well established experimental animals for the application of gene knock-out or transgenic modification technology are mice, and the application to large mammal animal models is still under exploration. Even there are some reports on the establishment of gene knock-out animal models of large mammal animals such as bovine, sheep, pig, monkey, etc, it is a great difficulty for in vitro manipulation of canine oocytes and the embryo, and thus the difficulties for establishing gene knockout or genetically modified model canines are increased due to the big difference of the breeding physiology between canines and other mammals. Thus, even there is a great amount of demand for diseases model canines having gene knock-out or transgenic modification, there are few reports on the successful establishment of diseases model canines having gene knock-out or transgenic modification worldwide. There is no report on model canines having atherosclerosis gene knock-out or transgenic modification at all.

Therefore, it is highly desirable to establish atherosclerosis gene knock-out or transgenic modification model canines, whose disease symptoms are primary symptoms. The disease phenotype thereof can last a long period of time, and the disease is hereditary, and consequently, offsprings of disease model canines can be obtained through natural reproduction. Thus, suitable experimental animal models having gene knock-out or transgenic modification can be provided for the study of cardiovascular diseases, and the research and development of related medicines.

### SUMMARY OF THE INVENTION

The present invention obtains a fertilized ovum or oocyte of APOE gene knock-out modified canine through gene knock-out technology, and the fertilized ovum or oocyte is then transplanted into one of the fallopian tubes of a female canine, of which both fallopian tubes have been embryo flushed to prepare an APOE gene knock-out canine.

In the first aspect, the present invention provides a method for establishing an APOE gene knock-out canine model, comprising the following steps: (1) obtaining a fertilized ovum or oocyte from APOE gene knock-out canine prepared by gene editing technology; and (2) transplanting the fertilized ovum or oocyte into one of fallopian tubes of a female canine, of which both fallopian tubes have been embryo flushed.

The gene editing technology in step (1) includes CRISPR, TALEN and ZFN.

In the second aspect, the present invention provides a method for establishing an APOE gene knock-out canine model, comprising the following steps: (1) determining a target site sequence directed to an exon sequence of canine APOE gene sequence; (2) synthesizing single-guide RNA (sgRNA) sequence and its complementary sequence according to the target site sequence determined in step (1), then linking the synthesized sequence with a skeleton vector to construct a sgRNA targeting vector; (3) in vitro transcribing the sgRNA targeting vector to obtain mRNA of the sgRNA, and in vitro transcribing CRISPR/Cas9 to obtain mRNA of CRISPR/Cas9; (4) mixing the mRNA of sgRNA and mRNA of CRISPR/Cas9 obtained in step (3), and then intracytoplasmic injecting the obtained mixture into the fertilized ovum or oocyte; and (5) transplanting the fertilized ovum or oocyte into one of fallopian tubes of a female canine, of which both fallopian tubes have been embryo flushed.

Preferably, the target site sequence is determined to direct to the sequences of exon 2 (SEQ ID NO: 1), exon 3 (SEQ ID NO: 2) or exon 4 (SEQ ID NO: 3). More preferably, the target site sequence is determined to direct to sequence of exon 3 (SEQ ID NO: 2).

Preferably, the target site sequence in step (1) is determined as follows:

(SEQ ID NO: 4)
5′-CCGGGTGGCAGACTGGCCAGCCC-3′.

Preferably, the sgRNA sequence and its complementary sequence synthesized in step (2) are as follows:

sgRNA sequence: ataGGGCTGGCCAGTCTGCCACCgt (SEQ ID NO: 5); and

the complementary sequence of sgRNA sequence: taaaacGGTGG CAGACTGGCCAGCC (SEQ ID NO: 6).

Preferably, the skeleton vector is T7-gRNA commercially available from Addgene.

Preferably, in step (5), the fertilized ovum or oocyte is transplanted into the less bleeding fallopian tube of a female canine, of which both fallopian tubes have been embryo flushed.

In the third aspect, in step (4) of the aforementioned second aspect, the mRNA of sgRNA and the mRNA of CRISPR/Cas9 obtained in step (3) are mixed, and is subsequently intracytoplasmic injected into a somatic cell, and the somatic cell nuclear is transplanted into an enucleated oocytes; in step (5) of the aforementioned second aspect, the enucleated oocyte is transplanted into one of the fallopian tubes of a female canine, of which both fallopian tubes have been embryo flushed.

In the fourth aspect, the present invention provides a canine APOE gene targeting vector, which consists of sgRNA sequence and its complementary sequence designed to direct to the target site sequence of the exons of canine APOE gene, and the skeleton vector.

Preferably, the exon is exon 2 (SEQ ID NO: 1), exon 3 (SEQ ID NO: 2) or exon 4 (SEQ ID NO: 3) of the canine APOE gene. Preferably, the skeleton vector is T7-gRNA commercially available from Addgene.

Preferably, the target site sequence is as follows:

(SEQ ID NO: 4)
5′-CCGGGTGGCAGACTGGCCAGCCC-3′.

Preferably, the sgRNA sequence and its complementary sequence are as follows:
sgRNA sequence: ataGGGCTGGCCAGTCTGCCACCgt (SEQ ID NO: 5); and

the complementary sequence of the sgRNA sequence: taaaacGGTGG CAGACTGGCCAGCC (SEQ ID NO: 6).

In the fifth aspect, the present invention provides a somatic cell, tissue or organ of APOE gene knock-out canine obtained through the method of any one of the aforementioned the first to the third aspects.

Preferably, the somatic cell comprises a sequence of cctggaccagggaggct (SEQ ID NO: 7).

Preferably, the somatic cell is ear fibroblast BGD-APOEKO-EFO of APOE gene knock-out beagle canine, which is deposited in China General Microbiological Culture Collection Center (CGMCC) on Mar. 1, 2017 with a CGMCC depository No. 13804.

In the sixth aspect, the present invention provides a primer pair for detecting APOE gene knock-out canine comprising a genomic sequence, further comprising a sequence fragment of cctggaccagggaggct (SEQ ID NO: 7), wherein the primer pair is designed to direct to the sequence of cctggaccagggaggct (SEQ ID NO: 7).

Preferably, the sequences of the primer pair are as follows:

Forward primer F:
(SEQ ID NO: 8)
5′-CATTGTTGTCAGGCAGGTAGC-3′;
and
Reverse primer R:
(SEQ ID NO: 9)
5′-GAAGGGTGCGAGGGATTGA-3′.

In the seventh aspect, the present invention provides a kit for detecting APOE gene knock-out canine comprising a genomic sequence, further comprising a sequence fragment of cctggaccagggaggct (SEQ ID NO: 7), wherein the kit comprises a primer pair designed to direct to the sequence of cctggaccagggaggct (SEQ ID NO: 7).

Preferably, the sequences of the primer pair are as follows:

Forward primer F:
(SEQ ID NO: 8)
5′-CATTGTTGTCAGGCAGGTAGC-3′;
and
Reverse primer R:
(SEQ ID NO: 9)
5′-GAAGGGTGCGAGGGATTGA-3′.

In the eighth aspect, the present invention provides an APOE gene knock-out canine obtained through the method of any one of the aforementioned the first aspect to the third aspect.

Canine APOE gene has total of four exons, and the translation initiation site is located within the second exon. The present invention carries out gene targeting at the third exon, resulting in frameshift mutation of the genomic sequence thereof, so that the translation is terminated at the 63^{rd} amino acid. Since the APOE protein cannot be fully expressed, thus the purpose of gene knock-out is achieved. Besides this site, gene targeting can also be carried out at any sequence of Exon 2, Exon 3 and Exon 4 of the canine APOE gene, causes changes of gene sequence and leads to advanced termination of amino acid translation. As a result, APOE protein cannot be fully expressed. Since incompletely expressed APOE protein cannot perform its original functions, thus the purpose of canine APOE gene knock-out can also be achieved.

The present invention prepares an APOE gene knock-out canine through choosing a target site sequence based on exons of the canine APOE gene sequence, constructing sgRNA targeting vector and CRISPR/Cas9 expression vector according to the target site sequence using gene editing, then intraplasmic injecting the mRNA of sgRNA and the mRNA of CRISPR/Cas9 obtained through in vitro transcription into a canine fertilized ovum, and transplanting the fertilized ovum into one of the fallopian tubes of a female canine, of which both fallopian tubes have been embryo flushed. This is the first time that APOE gene knock-out canine is successfully obtained in the world. Since both fallopian tubes have been embryo flushed prior to the transplantation, it increases numbers of transgenic fertilized ovum or oocyte and avoids the influence of embryos in the fallopian tube that was not embryo flushed on the implantation of the gene knock-out embryos, comparing to embryo flushing only one side of the fallopian tube. Thus, the utilization efficiency of the fertilized ovum and the survival rate of the transgenic canine are significantly increased.

In addition, the APOE gene knock-out canines obtained in the present invention will provide disease animal models of tremendous application value for medical research, and lay foundations for moving forward studying of cardiovascular disease and screening of cardiovascular disease drugs.

### Abbreviations and Key Terms Definitions

APOE: an apolipoprotein E, which is one of the apolipoproteins synthesized mainly in liver and brain tissue, and is a constituent of nervous system and plasma lipoproteins. APOE participates in metabolism process of cholesterol and triglyceride in blood by bonding low-density lipoprotein receptor to take in low-density lipoprotein. Human APOE gene is located at the long arm of the 19^{th} pair of chromosome and has a length of 37 kb. This gene comprises four exons and three introns. The cDNA thereof has a length of 1.63 kb, and the initiating product thereof is a protein comprising 317 amino acids. After being cleaved by a signal peptide comprising 18 amino acids, it becomes a mature protein consisting of 299 amino acids. The canine gene of APOE is located at number 1 chromosome of canine, and this gene has a total length of 2788 bp, which has total of four exons and six CDS regions for encoding proteins, and encode 323 amino acids.

ICI: intracytoplasmic injecting, which refers to injecting gene into cytoplasm of fertilized ovum through micromanipulation with use of a microinjection needle.

AS: atherosclerosis. Lipid metabolism disorder is the lesion foundation of atherosclerosis. The disease is characterized in that lesion of the involved arterial starts from endometrium. Accumulation of lipid and complex carbohydrate occurs at first in general, followed by bleeding and thrombosis. Hereafter, proliferation and calcinosis of fibrous tissue are further developed, and gradual metamorphosis and calcification of arterial media happen, which lead to thickening and hardening of arterial wall and vascular stenosis. The lesion often involves large and medium muscular artery. Once the lesion is developed enough to block the artery cavity, the tissue or organ supplied by the arteries will be ischemic or necrotic. Since the lipid accumulated in the arterial intimas is yellow atherosclerosis, it is called atherosclerosis.

### BRIEF DESCRIPTION OF THE DRAWINGS

FIG. 1 is a schematic diagram of the canine APOE gene target site sequence.

FIG. 2 shows Exon 3 sequence of the APOE gene of a wildtype canine and mutated sequence of Exon 3 sequence of the APOE gene of the gene knock-out canine, numbered 161207.

FIG. 3 shows sequence comparison of the APOE gene from different origins.

FIG. 4 shows a sequencing peak diagram of the APOE gene-edited canine.

### DETAILED DESCRIPTION OF THE INVENTION

Technical solutions of the present invention are further described in below through combining embodiments and drawings of the description. These embodiments are for illustrating rather than setting limit to the scopes of protection of the present invention.

### Embodiment 1: Constructing, In Vitro Transcription and Verification of Transgenic Targeting Vector

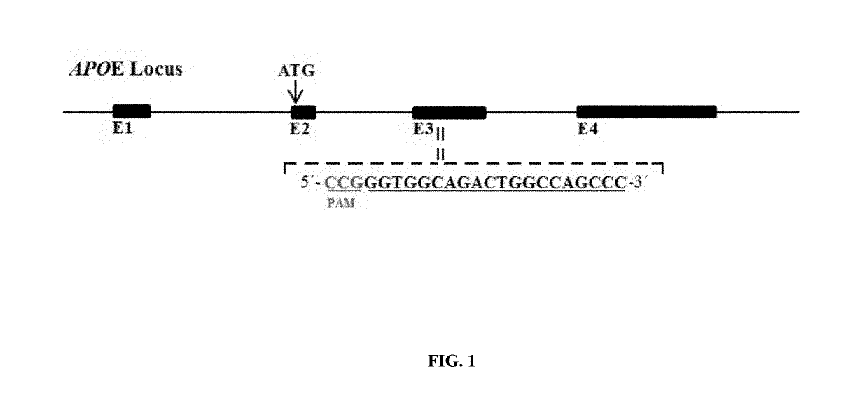

Choosing a target site sequence: 5′-CCGGGTGGCAGACTGGCCAGCCC-3′ (SEQ ID NO: 4) (see FIG. 1) based on Exon 3 of canine APOE gene according to canine APOE gene sequence information provided by NCBI. The sgRNA sequence identifying the target site sequence is 5′-GGGCTGGCCAGTCTGCCACC-3′ (SEQ ID NO: 10). When constructing a vector, the skeleton vector T7-gRNA (commercially available from Addgene) is enzyme digested with BbsI for subsequent experiments; sgRNA sequence: ataGGGCTGGCCAGTCTGC CACCgt (SEQ ID NO: 5) and sgRNA complementary sequence: taaaacGGTGGCAGACTGGCCAGCC (SEQ ID NO: 6) are designed; sgRNA sequence and the sgRNA complementary sequence are annealing linked, and then linked with enzyme digested T7-gRNA plasmid. A PCR product is recovered after PCR amplifying the T7-sgRNA plasmid, and an in vitro transcription kit is used for in vitro transcribing the PCR product of T7-sgRNA.

At first, the plasmid of CRISPR/Cas9 is linearized. The reacting system is as follows: 30 μg plasmid, 5 μl restriction endonuclease AflII; 10 μl of 10× Buffer and ddH₂O, the total volume is 100 μl. Then 100 μl of phenol: chloroform: isopropyl alcohol (25:24:1) is added to purify the linearized plasmid DNA, and then 12,000 g centrifugation for 5 min; sucking 50 μl supernatant into a 1.5 ml centrifugation tube without RNase, adding sodium acetate in 1/10 volume and anhydrous ethanol in 3 times volume to precipitate plasmid DNA, and then 12,000 g centrifugation for 5 min; discarding the supernatant, discard the remaining supernatant at the best; adding 150 μl of 70% ethanol to wash the plasmid, and then 12,000 g centrifugation for 5 min; drying in air for 3-5 min, dissolving DNA with 15 μl ddH₂O of RNase-free, and measuring the concentration.

In Vitro Transcription of mRNA with a Kit (Ambion):

The system of in vitro transcription is as follows: 1 μg linearized plasmid DNA, 10 μl 2×NTP/CAP, 2 μl 10× Buffer, 2 μl RNA synthetase and ddH₂O, total volume is 20 μl. After mixing homogeneously, incubating for 1 hr at 37° C.; adding 1 μl of TURBO DNA enzyme, digesting plasmid template, and incubating for 30 min at 37° C. Then, mixing 20 μl in vitro transcription product, 20 μl 10× Reaction Buffer, 10 μl ATP (10 mM), 2.5 μl RNase inhibitor, 2 μl Poly (A) polymerase and nuclease-free ddH₂O to form a system of in vitro transcription of poly (A) mRNA, with a total volume of 100 μl, incubating for 1 hr at 37° C. After incubation, adding 350 μl binding buffer to the reaction system and mixing homogeneously through blowing; adding 250 μl anhydrous ethanol, mixing homogeneously; then transferring the sample into an mRNA purification column, then 10,000 g centrifugation for 1 min at room temperature; discarding the filtrate, and then reloading the column, rinsing the column with 500 μl eluent, and then 10,000 g centrifugation for 1 min at room temperature; repeating the rinsing one time, discarding the filtrate, centrifugation of the empty column for 1 min to rinse off impurities such as proteins; then the column is placed into a new centrifugation tube, adding 50 μl RNA eluent to the central position of the column, covering the lid and incubating for 10 min at 65° C., then 10,000 g centrifugation for 1 min at room temperature; and measuring quality and concentration of the RNA.

The sgRNA of CRISPR and mRNA of Cas9 are mixed so that sgRNA has a final concentration of 20 ng/μl, and Cas9 has a final concentration of 200 ng/μl, storing at −80° C. for cytoplasmic injection.

The constructed sgRNA and Cas9 plasmid are co-transferred to canine skin fibroblasts, and G418 is used for screening. DNA is extracted from cell clones obtained by screening as a template, and the following primer pair is used to conduct PCR, which amplifies a DNA fragment of total 660 bp at the upstream and downstream of the target being identified and cleaved by sgRNA:

Forward primer F:
(SEQ ID NO: 8)
5′-CATTGTTGTCAGGCAGGTAGC-3′;
and
Reverse primer R:
(SEQ ID NO: 9)
5′-GAAGGGTGCGAGGGATTGA-3′.

The target fragment obtained through PCR amplification is subjected to DNA sequencing to determine the targeting efficiency of the vector. Total of 30 cell clones are obtained after transfection and screening, 26 cell clones have the gene mutation in the region of the target site showed by the PCR sequencing. The mutation efficiency is 86.7%, which proves that the constructed vector has a high accuracy, and the targeting efficiency is higher. Therefore, the constructed vector can be used for the preparation of APOE gene knock-out canine.

### Embodiment 2: Embryo Transplanting of APOE Gene Knock-Out Canine

Total of 13 female beagle canines of natural estrous are used as donors of fertilized ova and also receptors of embryo transplanting for experimental research. Blood sample is taken to measure progesterone level in serum. If the progesterone concentration is at 4-7 ng/ml, the ovulatory period is determined. Natural mating is performed after 48 hr of ovulation, and followed by flushing the fertilized ova. 65 fertilized ova are collected from 13 female canines. After the fertilized ova are collected, they are subjected to removing cumulus granulosa cells by using TCM199 medium comprising 0.1% hyaluronidase, followed by putting into droplets of HEPES buffered TCM199 medium (HM, GIBCO11150), and then placing on an inverted microscope equipped with a micromanipulator. A mixture comprising mRNA of sgRNA and mRNA of Cas9 prepared in Embodiment 1 at a ratio of 1:1 in volume is sucked with a microinjection needle, and then injected into cytoplasm of a fertilized ovum. The fallopian tube is flushed with 10 ml of HEPES buffered TCM199 medium (HM, GIBCO11150) comprising 10% fetal bovine serum, and the ovum flushing fluid is discharged from the injection needle ligated at umbrella of the fallopian tube, and is collected into a 10 ml centrifugation tube.

After the intraplasmic injection, the embryos are loaded into an embryo transplanting tube, and the embryos in the embryo transplanting tube are injected from the fallopian tube umbrella into the less bleeding one of fallopian tubes when embryo flushing.

TABLE 1
Embryo Transplanting Results

| | Number of | | Numbers of |
|---|---|---|---|
| No. of receptor | transplanted | Number of | gene knock-out |
| canines | fertilized ova | offspring | canine |
| FRA1115 | 8 | 0 | 0 |
| FRA1121 | 4 | 0 | 0 |
| FRA1126 | 5 | 0 | 0 |
| FRA1118 | 1 | 0 | 0 |
| FRA1124 | 5 | 4 | 1 |
| FRA1123 | 6 | 1 | 0 |
| FRA1129 | 7 | 1 | 0 |
| FRA1024 | 8 | 0 | 0 |
| FRA1130 | 6 | 2 | 0 |
| FRA1139 | 2 | 1 | 0 |
| FRA1137 | 2 | 6 | 1 |
| FRA1140 | 8 | 0 | 0 |
| FRA1146 | 3 | 1 | 0 |
| Total | 65 | 13 | 2 |

It can be seen from Table 1 that 13 female beagle canines transplanted with 65 fertilized ova produced a total of 13 offspring, and two of them are gene knock-out canines. Detection and verification are given in the following embodiments.

### Embodiment 3: Gene Mutation Detection of APOE Gene Knock-Out Canine

After the puppies are born, ear tissue and tail tissue are collected for identification. After the tissue block is fragmented in a centrifugation tube, protease K is added for water bath and cleavage at 56° C. for 1˜3 h. Then 700 μl of Genomic Lysis Buffer sucked with a pipette is added to the cleavage system, mixing homogeneously through turning upside down, and then 10000 g centrifugation for 1 min. The supernatant is sucked to a purifying column with a pipette, 10000 g, standing for 1 min at room temperature. A new collecting tube is used, and 200 μl DNA Pre-Wash Buffer is added to the centrifugation column, 10000 g, followed by standing for 1 min at room temperature, centrifuging for 1 min, and discarding wasted liquid. 400 μl g-DNA Wash Buffer is added to the centrifugation column, 10000 g, standing for 1 min at room temperature, centrifuging for 1 min, and discarding the wasted liquid. The purifying column and collecting tube are re-centrifuged, 10000 g, centrifuging for 2 min. The purifying column is placed in a newly replaced 1.5 ml centrifugation tube, 50 μl Elution Buffer is added to elute DNA, followed by standing for 2 min at room temperature, and then 12000 rpm, centrifuging for 1 min. The obtained solution is canine genomic DNA.

The canine genomic DNA is used as a template to carry out PCR, the primers are as follows:

Forward primer F:
(SEQ ID NO: 8)
5′-CATTGTTGTCAGGCAGGTAGC-3′;
and
Reverse primer R:
(SEQ ID NO: 9)
5′-GAAGGGTGCGAGGGATTGA-3′.

After amplification, a DNA fragment of total 660 bp at the upstream and downstream of the target being identified and cleaved by sgRNA. The target fragment obtained through PCR amplifying is undergone DNA sequencing, and is compared with canine APOE gene sequence provided by NCBI database to determine the mutation type of the APOE gene.

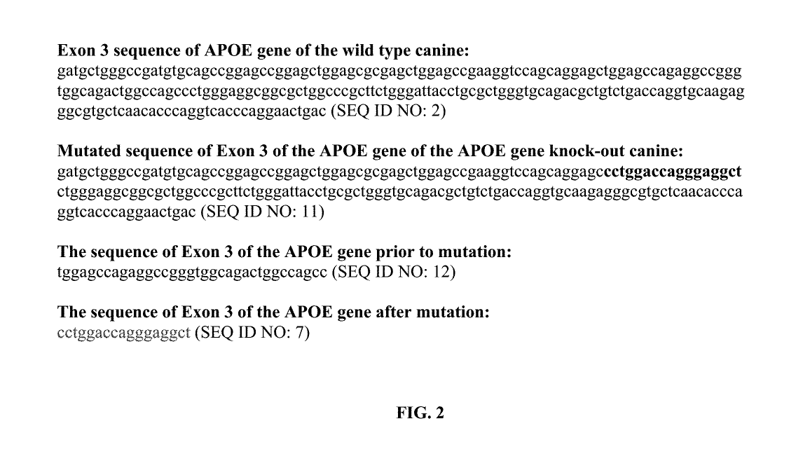

Upon sequencing and sequence alignment, it is demonstrated that among the 13 puppies, two puppies (one male and one female) have mutations in the Exon 3 target site of the APOE gene. The male canine (numbered 161207) has a deletion of a 34 bp fragment and an insertion of a 17 bp fragment, causing APOE gene homozygous double knock-out and resulting a mutated APOE protein starting from the 37^{th} amino acid and terminating at the 63^{th} amino acid; the female canine (numbered 170111) has a heterozygous mutation of deleting 33 bp at one side and deleting 51 bp at the other side. FIG. 2 shows Exon 3 sequence (SEQ ID NO: 2) of the APOE gene of a wildtype canine, and mutated sequence (SEQ ID NO: 11) of Exon 3 sequence of the APOE gene of the gene knock-out canine numbered 161207, which shows that a fragment of 34 bp was deleted, and a fragment of 17 bp was inserted at the same time. The bold part of Exon 3 sequence of APOE gene of the gene knock-out canine (numbered 161207) demonstrates the added fragment of 17 bp. Particularly, the sequence of the corresponding site prior to mutation is tggagccagaggccgggtggcagactggccagcc (SEQ ID NO: 12), and the sequence after the mutation is cctggaccagggaggct (SEQ ID NO: 7).

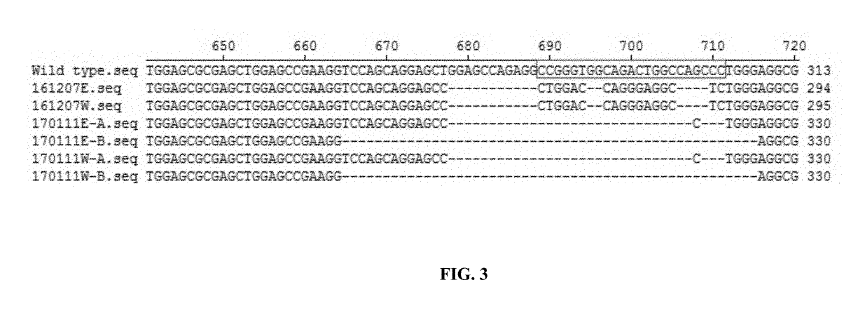

FIG. 3 shows the sequences alignment among the sequence of the 641^{st} to the 720^{th} nucleotide of Exon 3 of the APOE gene of a wildtype canine, the corresponding sequences of the Exon 3 from ear and tail tissues of APOE gene knock-out canine (numbered 161207), respectively, and the corresponding sequences of Exon 3 from ear and tail tissues of APOE gene knock-out canine (numbered 170111), respectively. Note that the target sequence is marked with a box; letter suffix E after the numeric number indicates ear tissue, suffix W after the numeric number indicates tail tissue; A and B indicate the number of alleles of APOE heterozygous knock-out canine, numbered 170111.

The Ear fibroblast BGD-APOEKO-EFO of APOE gene knock-out beagle canine numbered 161207 is deposited in China General Microbiological Culture Collection Center (CGMCC) on Mar. 1, 2017 with a CGMCC depository No. 13804.

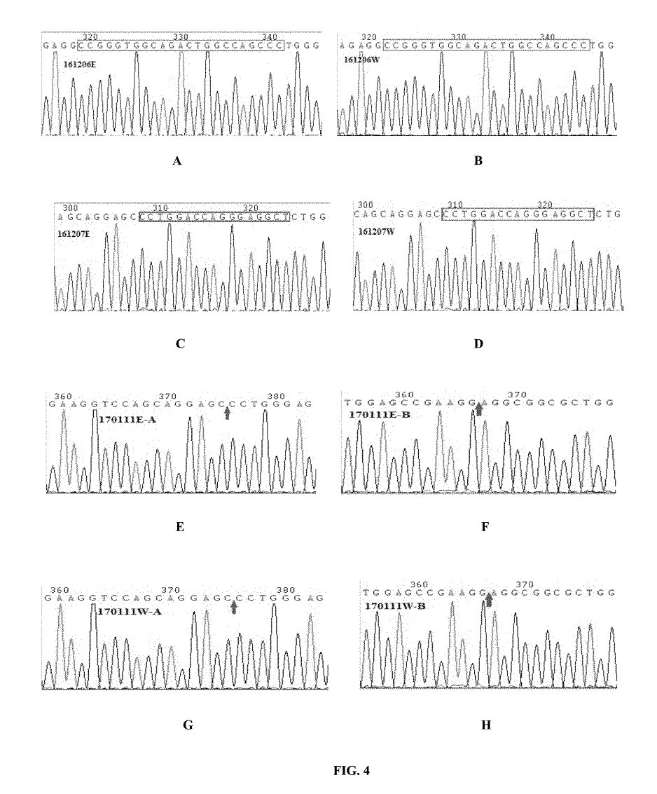

FIG. 4 shows a sequencing peak diagram of the APOE gene edited canine. The numbers in the figure are serial numbers of the canine, letter E indicates ear tissue, and letter W indicates tail tissue; 161206E/W indicates ear tissue and tail tissue of wildtype canine, respectively, and the target site region of the wildtype gene is marked with a box (see FIGS. 4A and 4B); 161207E/W indicates ear tissue and tail tissue of male canine having APOE gene mutation respectively, and the mutated sequence information is indicated in the box (see FIGS. 4C and 4D); 170111E/W indicates ear tissue and tail tissue of female canine having APOE gene mutation respectively, A and B are serial numbers of heterozygous mutation alleles, and the arrows point out the mutation region (see FIGS. 4E-4H).

### Embodiment 4: Blood Lipid Detection of the APOE Gene Knock-Out Canine

Blood is collected from three-month old APOE gene knock-out canine (161207), and is centrifuged for separating serum. The contents of total cholesterol, triglyceride, high-density lipoprotein and low-density lipoprotein in serum are measured. The results show that comparing with control canines (numbered 161205 and 161206, respectively), the contents of total cholesterol, triglyceride, high density lipoprotein and low density lipoprotein in serum of APOE gene knock-out canine are apparently higher than the control group (Table 2). It can be seen that the knock-out of APOE gene causes abnormal metabolism of lipids in the gene knock-out canine, leading to significant increase of blood lipid, which further verifies that the present invention obtains the APOE gene knock-out canine.

TABLE 2
Blood lipid detection result of the APOE gene knock-out canine

| | Serial No. | | |
|---|---|---|---|
| Items | 161207(APOE−/−) | 161205(WT) | 161206(WT) |
| Total | 22.92 | 7.225 | 8.25 |
| cholesterol (mmol/L) | | | |
| Triglycerides | 2.25 | 1.505 | 0.86 |
| (mmol/L) | | | |
| High density | 8.80 | 5.535 | 6.08 |
| lipoprotein (mmol/L) | | | |
| Low density | 13.10 | 1.15 | 1.78 |
| lipoprotein (mmol/L) | | | |

What is claimed is:

1. A method for establishing an APOE gene knock-out canine model, comprising the following steps: (1) obtaining a fertilized ovum or an oocyte from APOE gene knock-out canine prepared by gene editing technology; and (2) transplanting the fertilized ovum or the oocyte into one of the fallopian tubes of a female canine, of which both fallopian tubes have been embryo flushed.

2. The method according to claim 1, wherein the gene editing technology is CRISPR, TALEN or ZFN.

3. The method according to claim 1, further comprising the following steps: (1) determining a target site sequence directed to an exon sequence of canine APOE gene; (2) synthesizing sgRNA sequence and its complementary sequence according to the target site sequence determined in step (1), linking the synthesized sequence with a skeleton vector to construct a sgRNA targeting vector; (3) in vitro transcribing the sgRNA targeting vector to obtain mRNA of the sgRNA, in vitro transcribing CRISPR/Cas9 to obtain mRNA of CRISPR/Cas9; (4) mixing the mRNA of the sgRNA and the mRNA of CRISPR/Cas9 obtained in step (3), intracytoplasmic injecting the obtained mixture into the fertilized ovum or oocyte; and (5) transplanting the fertilized ovum or oocyte into one of the fallopian tubes of a female canine, of which both fallopian tubes have been embryo flushed.

4. The method according to claim 3, wherein the target site sequence is determined to direct to the sequences of exon 2 (SEQ ID NO: 1), exon 3 (SEQ ID NO: 2) or exon 4 (SEQ ID NO: 3).

5. The method according to claim 3, wherein the target site sequence in step (1) is the sequence selected to direct to exon 3 (SEQ ID NO: 2) as follows:
(SEQ ID NO: 4)
5′-CCGGGTGGCAGACTGGCCAGCCC-3′.

6. The method according to claim 3, wherein the synthesized sgRNA sequence and its complementary sequence in step (2) are as follows:
sgRNA sequence: ataGGGCTGGCCAGTCTGCCACCgt (SEQ ID NO: 5); and
complementary sequence of the sgRNA sequence: taaaacGGTGG CAGACTGGCCAGCC (SEQ ID NO: 6).

7. The method according to claim 3, wherein in step (5), the fertilized ovum or oocyte is transplanted into the less bleeding fallopian tubes of a female canine, of which both fallopian tubes have been embryo flushed.

8. The method according to claim 1, further comprising the following steps: (1) determining a target site sequence directed to an exon sequence of the canine APOE gene sequence; (2) synthesizing sgRNA sequence and its complementary sequence according to the target site sequence determined in step (1), linking the synthesized sequence with a skeleton vector to construct a sgRNA targeting vector; (3) in vitro transcribing the sgRNA targeting vector to obtain mRNA of the sgRNA, in vitro transcribing CRISPR/Cas9 to obtain mRNA of CRISPR/Cas9; (4) mixing the mRNA of the sgRNA and the mRNA of CRISPR/Cas9 obtained in step (3), intracytoplasmic injecting the obtained mixture into canine somatic cells, nuclear transplanting the somatic cells into a canine enucleated oocyte; and (5) transplanting the canine enucleated oocyte into one of the fallopian tubes of a female canine, of which both fallopian tubes have been embryo flushed.

9. A canine APOE gene targeting vector, consisting of an sgRNA sequence and its complementary sequence directed to a target site sequence of an exon of the canine APOE gene, and a skeleton vector.

10. The gene targeting vector according to claim 9, wherein the exon of the canine APOE gene is exon 2 (SEQ ID NO: 1), exon 3 (SEQ ID NO: 2) or exon 4 (SEQ ID NO: 3) thereof.

11. The gene targeting vector according to claim 9, wherein the target site sequence is selected as follows: 5′-CCGGGTGGCAGACTGGCCAGCCC-3′ (SEQ ID NO: 4).

12. The gene targeting vector according to claim 9, wherein the sgRNA sequence and its complementary sequence are as follows:
sgRNA sequence: ataGGGCTGGCCAGTCTGCCACCgt (SEQ ID NO: 5); and
complementary sequence of the sgRNA sequence: taaaacGGTGG CAGACTGGCCAGCC (SEQ ID NO: 6).

13. Ear fibroblast BGD-APOEKO-EFO of APOE gene knock-out beagle canine, which is deposited in China General Microbiological Culture Collection Center (CGMCC) on Mar. 1, 2017 with a CGMCC depository No. 13804.

14. A primer pair for detecting APOE gene knock-out canine, comprising a genomic sequence comprising a sequence fragment as shown by cctggaccagggaggct (SEQ ID NO: 7), wherein the primer pair is designed to direct to the sequence as shown by cctggaccagggaggct (SEQ ID NO: 7).

15. The primer pair according to claim 14, wherein the sequences of the primer pair are as follows:
Forward primer F:
(SEQ ID NO: 8)
5′-CATTGTTGTCAGGCAGGTAGC-3′;
and
Reverse primer R:
(SEQ ID NO: 9)
5′-GAAGGGTGCGAGGGATTGA-3′.
